(19) United States
(12) Patent Application Publication
(10) Pub. No.: US 2023/0203014 A1
(43) Pub. Date: Jun. 29, 2023

(54) NOVEL FLUORESCENT COMPOUNDS FOR LABELING TUMOR TISSUE

(71) Applicant: PROIMAGING, PARIS (FR)

(72) Inventors: Joanne TRAN-GUYON, MONTIGNY LE BRETONNEUX (FR); Vincent GUYON, MONTIGNY LE BRETONNEUX (FR); François SCHERNINSKI, PARIS (FR)

(73) Assignee: PROIMAGING, PARIS (FR)

(21) Appl. No.: 17/925,434

(22) PCT Filed: May 12, 2021
(86) PCT No.: PCT/FR2021/050832

§ 371 Date: Nov. 15, 2022

(30) Foreign Application Priority Data
May 15, 2020 (FR) FR2004868

(51) Int. Cl. C07D 403/08; A61K 49/00

(52) U.S. Cl. CPC C07D 403/08; A61K 49/0032

(57) ABSTRACT

Novel fluorescent compounds that can be used for labelling tumour tissue, and the method for labelling tumour tissue the novel fluorescent compounds. Also, the application of the tumour tissue labelled with the novel fluorescent compounds as a monitoring tool, a diagnostic tool, or a tool for assisting with cancer surgery.

The present invention relates to novel fluorescent compounds usable for labeling tumor tissue, the method of preparation thereof, as well as the application thereof as a tool for monitoring, diagnosis or as an aid in cancer surgery.

### FIELD

Labeling of tumor tissue with fluorescent compounds is of considerable interest in the field of medical imaging, because among other things it allows the localization of tumors.

### BACKGROUND

Fluorescent compounds have been used for more than fifty years in medicine as markers in noninvasive imaging techniques for surveillance and/or diagnosis.

### TECHNICAL PROBLEM

The emergence of new fluorescent imaging technologies in the service of surgery, requiring improved sensitivity and accuracy, leads to research for new fluorescent molecules giving better and better performance.

In the context of diseases such as cancer, it is necessary in particular to have a preferential distribution of the fluorescent molecules in the tumor tissues relative to the healthy tissues, as well as sufficiently long persistence of the fluorescence to allow improved specificity of labeling and to supply an aid for surgical intervention, for example to delimit the regions of tumors to be removed.

Certain existing fluorescent markers have limited persistence of fluorescence, which necessitates operating on the patient soon after injection of the marker and does not allow a satisfactory delimitation of the tumor tissue to be obtained. In other cases, there is insufficient accumulation of the marker in the tissues, which leads to poor labeling and therefore problems in detection. The localization of lesions or of tumors and then their removal for example by surgery is then incomplete.

Another problem with the existing fluorescent markers, in particular indocyanine green (ICG), which is one of the few dyes used for labeling tumors during a surgical procedure, is the need for the presence of tumoral neovascularization to obtain labeling of the tumor tissue. Moreover, indocyanine green, like the other existing dyes in the prior art, is only visible in tumor tissues up to 24h after its injection. This short duration does not give good elimination of the circulating dye outside of the tumor tissues, which causes poor visualization as the signal/noise ratio is low.

Another major drawback of the existing compounds is that they cannot be used directly. In fact, in order to be used they need to be coupled with other targeting molecules such as antibodies, proteins, molecules specific to the tumor tissue, folic acid, or steroids.

The present invention allows us to overcome the problems of the prior art explained above by supplying fluorescent molecules having preferential distribution in the tumor tissues relative to the healthy tissues and sufficient persistence for use thereof in imaging techniques for monitoring, diagnosis, and/or as an aid in surgery. These new molecules have the major advantage that they can be used alone and directly, without prior coupling, owing to their specific affinity for tumor tissue. Moreover, relative to the molecules of the prior art, they remain in the tumor tissues much longer, for up to several days, which allows more thorough elimination of these fluorescent molecules circulating outside of the tumor tissues, and thus improved visualization owing to a better signal/noise ratio.

### SUMMARY

The present invention relates to a compound of formula (I)

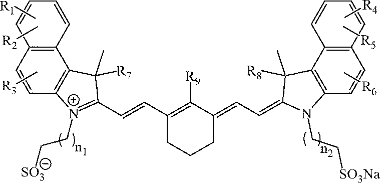

(I)

in which
n₁ and n₂ are each an integer from 0 to 15,
R₁, R₂, R₃, R₄, R₅ and R₆ are each selected independently from H, OH, SH, NH₂, SO₃R₁₀ and X—R₁₁—Y,
R₁₀, R′₁₀ being independently H, Na or K,
X, X′, X″ being independently O, S or NH,
R₁₁, R′₁₁, R″₁₁ being selected independently from C₁ to C₁₅ alkyl, aryl, heteroaryl, (C₁ to C₁₅ alkyl)aryl, (C₁ to C₁₅ alkyl)heteroaryl, aryl(C₁ to C₁₅ alkyl) and heteroaryl(C₁ to C₁₅ alkyl);
Y, Y′, Y″ being selected independently from H, halogen, COOR′₁₀ or amide;
R₇ and R₈ each being selected from H, OH, SH, NH₂, C₁ to C₁₅ alkyl, and X′—R′₁₁—Y′; R₉ being selected from H, OH, SH, NH₂ and X″—R″₁₁—Y″,
said compound comprising at least one group X—R₁₁—Y, X′—R′₁₁—Y′ or X″—R″₁₁—Y″ with Y, Y′, and/or Y″ which is COOR′₁₀.

The present invention also relates to the method of preparation of the compounds of formula (I) according to the invention, as well as the method for labeling tumor tissue with one of the compounds according to the invention or prepared according to the method of the invention.

### BRIEF DESCRIPTION OF THE FIGURES

FIG. 1 shows the median values and standard deviations of the tumor/abdomen intensity ratios as a function of time post-injection of compound 2 (CJ215) and of ICG.

FIG. 2 shows the results of ex vivo imaging of pancreatic tumors after injection of two compounds according to the invention and of a fluorescent agent of the prior art (ICG).

### DETAILED DESCRIPTION

The present invention relates firstly to a compound of formula (I)

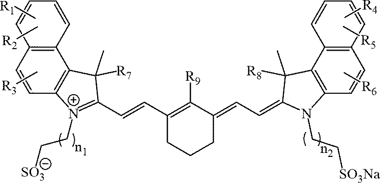

(I)

in which
n₁ and n₂ are each an integer from 0 to 15,
R₁, R₂, R₃, R₄, R₅ and R₆ are each selected independently from H, OH, SH, NH₂, SO₃R₁₀ and X—R₁₁—Y,
R₁₀, R′₁₀ being independently H, Na or K,
X, X′, X″ being independently O, S or NH,
R₁₁, R′₁₁, R″₁₁ being selected independently from C₁ to C₁₅ alkyl, aryl, heteroaryl, (C₁ to C₁₅ alkyl)aryl, (C₁ to C₁₅ alkyl)heteroaryl, aryl(C₁ to C₁₅ alkyl) and heteroaryl(C₁ to C₁₅ alkyl);
Y, Y′, Y″ being selected independently from H, halogen, COOR′₁₀ or amide;
R₇ and R₈ each being selected from H, OH, SH, NH₂, C₁ to C₁₅ alkyl, and X′—R′₁₁—Y′; R₉ being selected from H, OH, SH, NH₂ and X″—R″₁₁—Y″,
said compound comprising at least one group X—R₁₁—Y, X′—R′₁₁—Y′ or X″—R″₁₁—Y″ with Y, Y′, and/or Y″ which is COOR′₁₀.

In the sense of the present invention, “C₁ to C₁₅ alkyl” means a cyclic, linear, or branched hydrocarbon chain containing from 1 to 15 carbon atoms, preferably from 2 to 6 carbon atoms and even more preferably from 4 to 6 carbon atoms, in particular 5 carbon atoms and that may in particular be a methyl, ethyl, n-propyl, isopropyl, n-butyl, isobutyl, sec-butyl, tert-butyl, n-pentyl, 1-methylbutyl, 2,2-dimethylbutyl, 2-methylpentyl, 2,2-dimethylpropyl, isopentyl, neopentyl, 2-pentyl, hexyl, 2-hexyl, 3-hexyl, 3-methylpentyl, heptyl, octyl, nonyl, decyl, dodecyl, or palmityl chain.

In the sense of the present invention, “aryl” means an aromatic group, containing one or more aromatic rings, optionally substituted.

In the sense of the present invention, “heteroaryl” means an aromatic group, containing one or more aromatic rings, optionally substituted, and comprising at least one heteroatom different than carbon and hydrogen.

In the sense of the present invention, “aralkyl” means an aryl group substituted with one or more alkyl groups; said alkyl groups may be C₁ to C₁₅ alkyl groups, preferably containing from 1 to 15 carbon atoms.

In the sense of the present invention, “heteroaralkyl” means a heteroaryl group substituted with one or more alkyl groups; said alkyl groups may be C₁ to C₁₅ alkyl groups, preferably containing from 1 to 15 carbon atoms.

According to a particular embodiment, in the above formula, n₁ or n₂ are, independently of one another, equal to 1, 2, 3, 4 or 5, even more preferably 3 or 4.

According to another particular embodiment, in the above formula n₁=n₂ and is preferably equal to 1, 2, 3, 4 or 5, even more preferably 3 or 4.

According to another particular embodiment, the molecule is symmetric. In this case, it comprises a single group X″—R″₁₁—Y″ with Y″ which is COOR′₁₀ that is carried by R₉ and/or 2 groups X—R₁₁—Y with Y which is COOR′₁₀, one carried by one of R₁, R₂, R₃ or R₇, preferably one of R₁, R₂ or R₃ and the other carried by one of R₄, R₅, R₆ or R₈, preferably one of R₄, R₅ or R₆.

According to a particular embodiment, in the above formula, R₁₀, and/or R′₁₀, may be identical. Similarly, X, X′, and/or X″ may be identical, Y, Y′, and/or Y″ may be identical, R₁₁, R′₁₁, and/or R″₁₁ may be identical.

The compounds according to the invention may in particular be selected from the compounds of the following general formula:

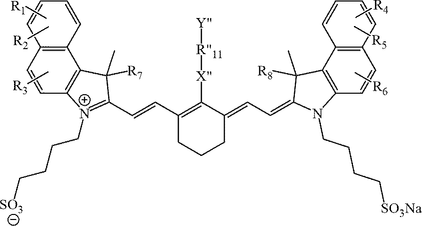

for which X″ may be O, S or NH, corresponding to the following formulas:

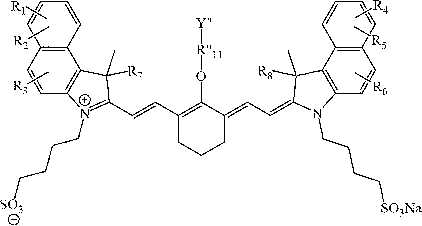

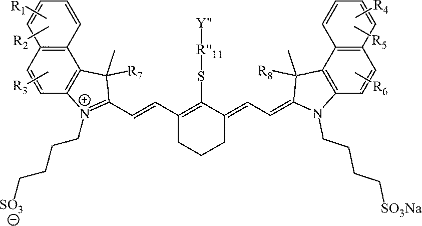

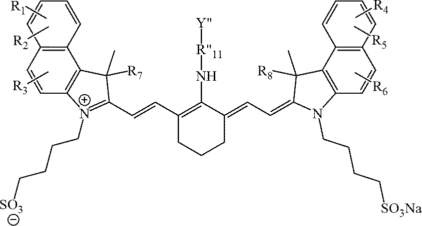

The compounds of formula (I) according to the invention may in particular be selected from the compounds of formula (I) for which R₁, R₂, R₃, R₄, R₅ and R₆ are not all simultaneously H, which excludes in this case the compounds according to the following formula (II):

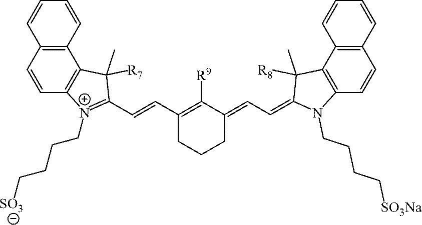

The compound of formula (I) according to the invention may preferably be selected from the compounds of the following general formulas:

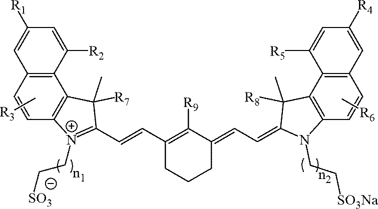

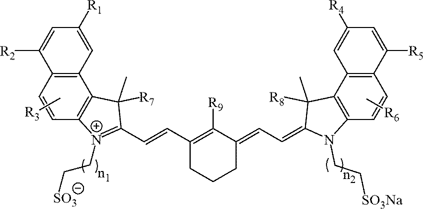

in which R₁, R₂, R₃, R₄, R₅, R₆, R₇, R₈ and R₉ are as defined above.

According to a particular embodiment, the compounds according to the invention may be selected from the compounds of the following formulas

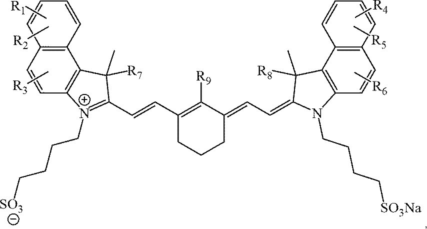

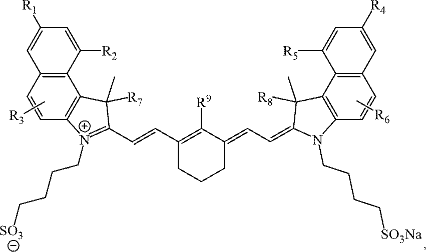

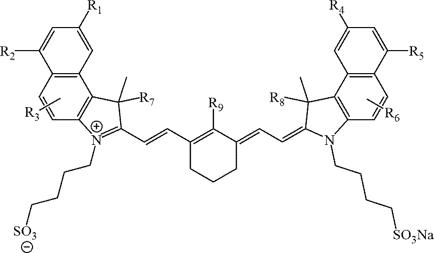

in which R₁, R₂, R₃, R₄, R₅, R₆, R₇, R₈ and R₉ are as defined above.

According to a preferred embodiment, the compounds according to the invention may be selected from the following compounds:

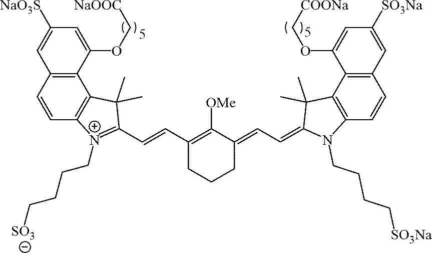

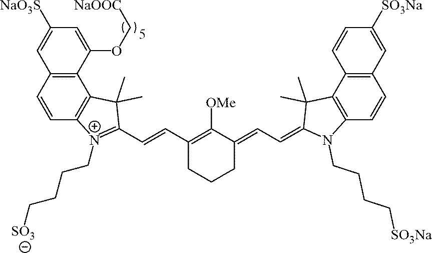

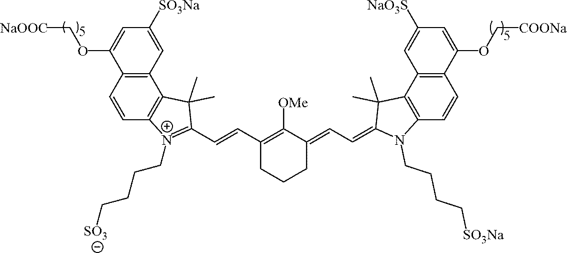

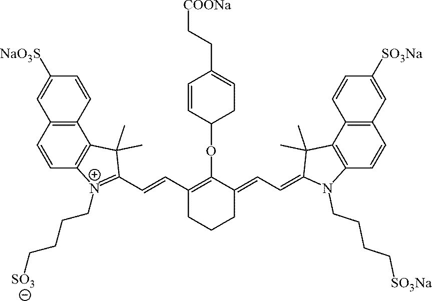

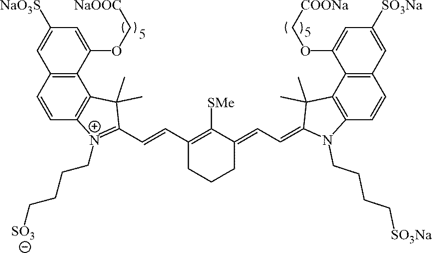

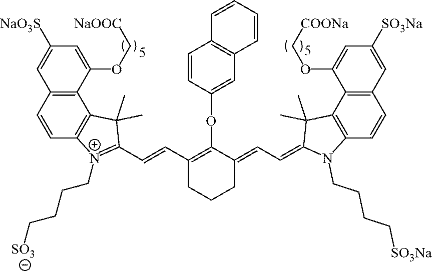

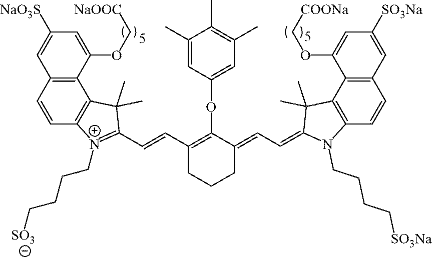

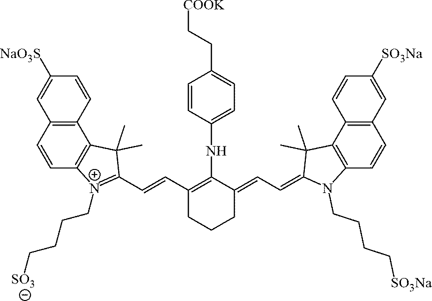

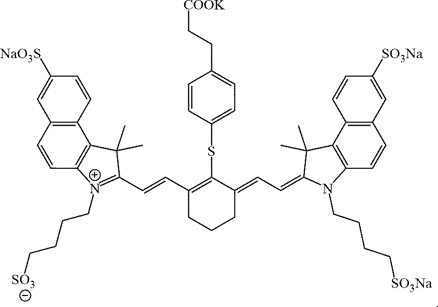

The present invention relates secondly to the method of preparation of the compounds of formula (I) according to the invention comprising a reaction step between:

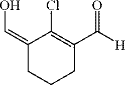 [SMILES: O=CC1=C(Cl)/C(=C\O)CCC1]

and

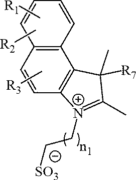

This reaction is preferably carried out by heating under reflux in the presence of sodium acetate in a mixture of acetic acid and acetic anhydride.

The invention relates thirdly to the method for labeling tumor tissue with one of the compounds according to the invention or prepared according to the method of the invention.

In the sense of the present invention, “tumor tissue” means tissue consisting of tumor cells, which are abnormal proliferating cells, and of a supporting tissue, also called tumoral stroma or interstitial tissue, composed of cells and extracellular substance in which the tumoral vascularization is located.

The fluorescent compounds according to the invention have the particular feature, after they have diffused in the body, of being trapped in tumor tissue, whereas they are eliminated from healthy tissues. This particular feature makes it possible to use these fluorescent compounds directly, without prior coupling to another labeling molecule, thus making their use simpler, quicker and more effective than that of the compounds of the prior art. It was observed that this elimination from healthy tissues increases over time. In general, between 24 and 72 hours, preferably between 36 and 60 hours and more preferably 48 hours after administration of these compounds, their elimination from healthy tissues is total. However, they remain trapped in the tumor tissues. This property gives a clear differentiation of tumor tissues relative to healthy tissues and thus these compounds can be used in applications of monitoring, diagnosis and/or as an aid to surgery in a context of cancerous diseases. This differentiation lasts for 6 to 48 hours, preferably 12 to 36 hours, allowing targeted programming of diagnosis or surgery.

The compounds according to the invention may thus be used in particular in the context of cancers, for example hormone-dependent cancers, such as breast cancer or digestive system cancers, such as pancreatic cancer. In fact, in pancreatic cancer, the tumors are particularly difficult to remove completely by surgery, as they are not easily delimited. The use of the compounds according to the invention makes it possible to obtain better visualization of the contours of the tumors owing to the differentiation of labeling between tumor tissue and healthy tissue, and thus more effective tumor resection by surgery.

The invention also relates to the use of one of the compounds according to the invention or prepared according to the method of the invention in a method for labeling tumor tissue.

This method of labeling tissues requires administration of the compounds by the intravenous or intraarterial route, or in another vessel, in particular a lymphatic vessel, or by local injection, or by local application, preferably by the intravenous route.

The invention further relates to a composition comprising one of the compounds according to the invention or prepared according to the method of the invention and at least one pharmaceutically acceptable adjuvant.

The invention also relates to one of the compounds according to the invention or prepared according to the method of the invention or a composition comprising one of the compounds according to the invention or prepared according to the method of the invention for use thereof in a method of labeling and/or detection of tumor tissue, and/or in the surgical treatment of tumors.

The invention also relates to a method for detecting tumor tissue comprising a step of labeling tumor tissue with one of the compounds according to the invention or prepared according to the method of the invention, and a step of detection by medical fluorescence imaging or fluorescence spectrometry.

### EXAMPLES

### Example 1

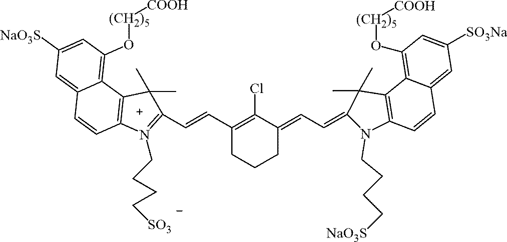

### Compound (1)

A mixture of 4-[(5-carboxypentyl)oxy]-6-sulfo-1-(4-sulfobutyl)-2,3,3-trimethyl-benz(e)indolium (internal salt and disodium salt) (9 g; 15 mmol), 2-chloro-1-formyl-3-(hydroxymethylene)-1-cyclohexene (1.30 g; 7.50 mmol), and sodium acetate (3 g; 36.6 mmol) in a 60/30 mixture of acetic acid and acetic anhydride is heated under reflux for 10 minutes. The reaction mixture is cooled to room temperature and the precipitate is separated by filtration and washed with diethyl ether to give 4.33 g (yield: 43.9%) of a green solid. The crude product is purified by column flash chromatography (inverse phase silica gel C18, acetonitrile 0-25% / water).

### Example 2

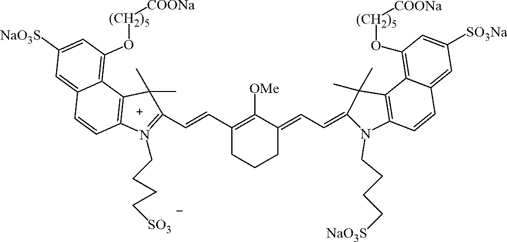

### Compound (2)

Sodium methylate (440 mg; 7.6 mmol) is added to compound (1) (1 g; 0.76 mmol) in solution in 500 mL of methanol. The reaction mixture is heated under reflux for 16 h and concentrated under vacuum, and then filtered. The residue obtained is washed with cold methanol and with acetone and dried under vacuum to give 450 mg of a green solid (yield: 45%). The crude product is purified by column flash chromatography (inverse phase silica gel C18, acetonitrile 0-25% / water).

### Example 3

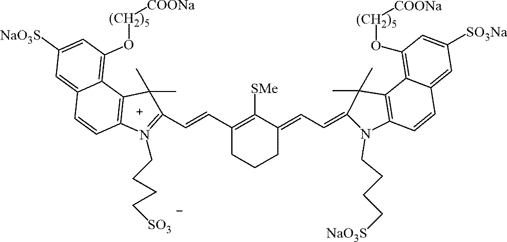

### Compound (3)

MeSNa (106 mg; 1.5 mmol) is added to compound (1) (400 mg; 0.30 mmol) in solution in 20 mL of a 50/50 mixture of methanol/NMP (N-methyl-2-pyrrolidone). The reaction mixture is heated under reflux for 4 h, and then diethyl ether (20 mL) is added to the mixture. The precipitate is filtered and washed with the same solvent to give 254 mg of crude product (yield: 61%; sulfur odor). The crude product is purified by column flash chromatography (inverse phase silica gel C18, acetonitrile 0-25% / water).

### Example 4

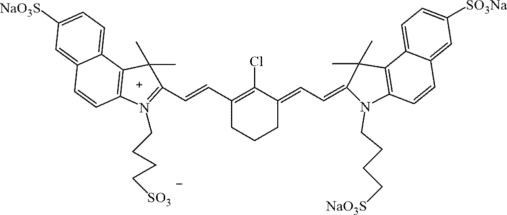

### Compound (4)

A mixture of 6-sulfo-1-(4-sulfobutyl)-2,3,3-trimethylbenz(e)indolium (internal salt and DCHA salt) (2 g; 4.7 mmol), 2-chloro-1-formyl-3-(hydroxymethylene)-1-cyclohexene (0.40 g; 2.35 mmol), and sodium acetate (0.9 g; 11 mmol) in a 50/20 mixture of acetic acid and acetic anhydride is heated under reflux for 15 minutes. The precipitate is separated by filtration, washed with ethanol and acetone and dried under vacuum to give 1.6 g (yield: 63.8%) of a brick-red powder. The crude product is purified by column flash chromatography (inverse phase silica gel C18, acetonitrile 0-25% / water).

### Example 5

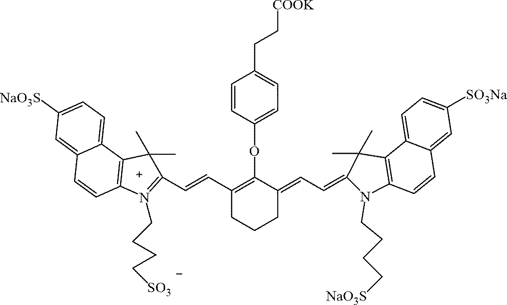

### Compound (5)

8 mL of 1 M methanolic KOH, 16 mL of DMSO and compound (4) (500 mg; 0.25 mmol) are added to 3-(4-hydroxyphenyl)propionic acid (660 mg; 4 mmol). The reaction mixture is stirred at room temperature for 8h, and then 150 mL of ethyl acetate is added dropwise. The precipitate is separated by filtration, washed with ethanol and acetone and dried under vacuum to give 260 mg (yield: 45%) of a green powder. The crude product is purified by column flash chromatography (inverse phase silica gel C18, acetonitrile 0-25% / water).

### Example 6

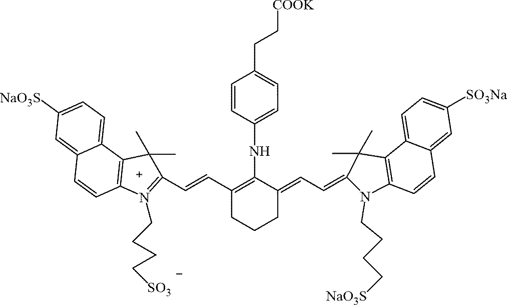

### Compound (6)

4-Aminohydrocinnamic acid (816 mg; 4.9 mmol), 25 mL of DMSO and triethylamine (500 mg; 4.9 mmol) are added to compound (4) (520 g; 0.49 mmol). The reaction mixture is stirred at room temperature for 8 h, and then 200 mL of acetone is added dropwise. The precipitate is separated by filtration, washed with acetone and dried under vacuum to give 430 mg (yield: 74%) of a red powder. The crude product is purified by column flash chromatography (inverse phase silica gel C18, acetonitrile 0-25% / water).

### Example 7

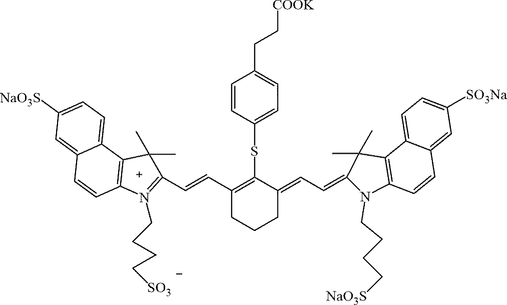

### Compound (7)

1 mL of 1 M methanolic KOH, 16 mL of DMSO and compound (4) (500 mg; 0.25 mmol) are added to 4-mercaptohydrocinnamic acid (91 mg; 0.5 mmol). The reaction mixture is stirred at room temperature for 30 minutes, and then 50 mL of ethyl acetate is added dropwise. The precipitate is separated by filtration, washed with ethanol and acetone and dried under vacuum to give 310 mg (yield: 54%) of a green powder. The crude product is purified by column flash chromatography (inverse phase silica gel C18, acetonitrile 0-25% / water).

### Example 8: Comparison of a Compound According to the Invention and A Fluorescent Agent of the Prior Art (ICG) for in Vivo Imaging of Mammary Tumors

ICG (or indocyanine green / Infracyanine) is a fluorescent agent of the prior art, already approved for use in humans for evaluation of cardiac and hepatic function, as well as in ophthalmology, for retinal diseases. It is also undergoing evaluation in many clinical trials throughout the world for guidance of surgery during tumoral exereses, or mapping of the ganglia draining the tumors, by near infrared imaging. ICG was compared with compound (2) according to the invention, synthesis of which is described above in example 2; this compound is called CJ215 in this study.

The study included 30 mice in total, distributed in three groups. The tumoral grafts were all carried out with 50 000 cells 4T1-Dendra2 /20 µl injected in 2 mammary glands contralaterally for each of the mice.

The injections of the biomarkers (compound 2 called CJ215 in this study and ICG) were carried out on D9 post-tumoral grafting (to limit the appearance of necrosis in the tumors).

The variation of the intensity of the fluorescence signals recorded for each of the biomarkers over time was evaluated from the microscopy image. The capacity of the two markers for producing a signal specifically localized to the tumor was assessed quantitatively by calculating the ratio of the specific signal associated with the tumor to the nonspecific signal in the surrounding tissues.

The imaging protocol was carried out at times 2 h, 24 h, 48 h, 4 and 6 days post-injection for all the mice. All the images made at each acquisition time were acquired on the IVIS Spectrum imager (Perkin Elmer) with the following parameters:
For detection of the GFP form of Dendra2 (detection of the tumor):
   Excitation at 465 nm
   Emission between 520 and 580 nm
For detection of the biomarkers:
   Excitation at 745 nm
   Emission between 800 and 840 nm

The quantitative measurements were performed on the raw images, which had not been deconvoluted. For the two fluorophors, the acquisition time was parameterized in automatic mode. In this mode, the system determines the acquisition time taken to reach the stipulated target value (6000 counts) in the time allowed (fixed at 2 min).

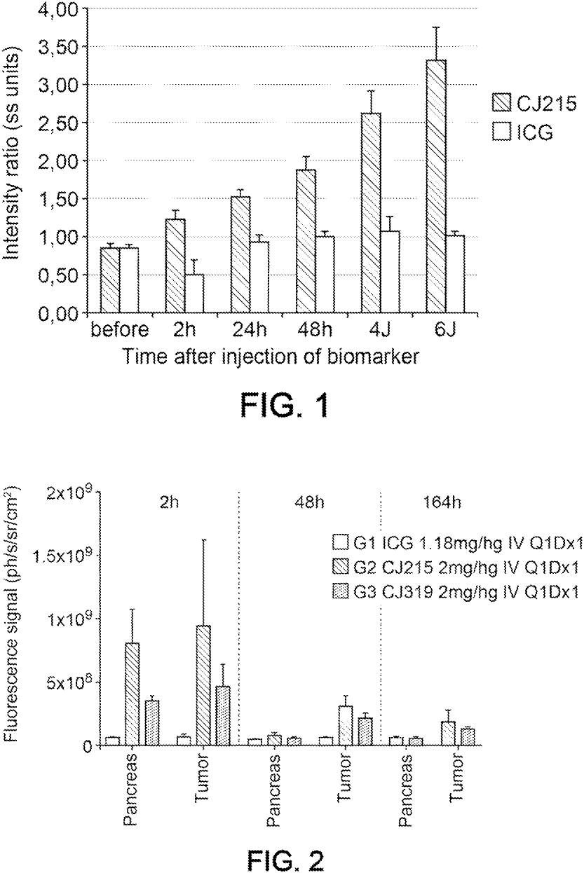

FIG. 1 reports the median values and standard deviations of the tumor/abdomen intensity ratios as a function of time post-injection of CJ215 and ICG.

Measurement of the ratio of the tumor/abdomen intensities illustrated in this figure makes it possible to show that:
the intensity ratios are significantly higher for compound 2 (CJ215) compared to ICG, regardless of the time post-injection (from 1.5 times at 2 h to more than 3 times at D+6), which translates into a capacity for identifying a specific signal in the tumors earlier and more specifically with compound 2 (CJ215). These results also indicate the possibility of very significantly improving the specificity of the signal to the tumor by increasing the time between injection of compound 2 (CJ215) and imaging;
the signal/noise ratio increases continuously for compound 2 (CJ215) up to 6 days post-injection, the last day of examination considered in this protocol. At this stage, ICG is no longer observable in the tumors (starting from 48 h). The great stability of the intratumoral signal of compound 2 (CJ215), compared to the surrounding tissues, which eliminate the product, thus offers an improved capacity for identifying the tumors and thus contributes to significant improvement in the fine delimitation of the tumoral margins, which is still problematic with ICG.

### Example 9: Comparison of Two Compounds According to the Invention And a Fluorescent Agent of the Prior Art (ICG) in ex Vivo Imaging of Pancreatic Tumors

A model of orthotopic pancreatic adenocarcinoma in the mouse was developed. The tumoral cells were amplified subcutaneously in SCID mice and the resultant fragments were then implanted surgically in the pancreas of irradiated BALB/c nude mice.

The development of the tumor was monitored in vivo by MRI (4.7T, PharmaScan, Bruker Biospin) at three time points, D14, 28 and 36. The animals were subjected to a weak fluorescence in order to minimize autofluorescence. Fluorescent imaging was performed with a charge-coupled device (CCD) camera (PhotonRT, BiospaceLab) with excitation at 700 nm and an emission filter at 770 nm.

After sessions of in vivo imaging performed at 2 h, 48 h and 164 h, ex vivo fluorescent images were acquired. The fluorescent compounds according to the invention 2 (CJ215) and CJ319 (the structure of which is detailed below) were injected intravenously at 2 mg/kg, 39 days after implantation of the tumor fragments, whereas the average volumes of the tumors were about 70 mm³. Indocyanine green (ICG), a dye widely used in the per-operative imaging of tumors, was included as a control.

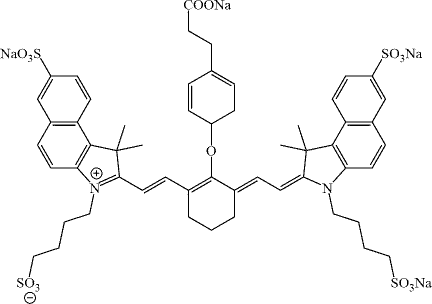

(CJ319)

The ex vivo fluorescence imaging described in FIG. 2 showed that 2 hours after injection, the two fluorescent compounds according to the invention were present in the pancreas and the tumor in almost equivalent amounts. However, 48 hours after injection, a clear preferential distribution was observed for the tumor, both compounds producing a fluorescent signal about four times higher in the tumor than in the surrounding pancreatic tissue. This effect persisted six days after injection, although the signal decreased as time passed. In comparison, indocyanine green did not show any specific accumulation in the pancreas or the tumor.

These results show the superiority of the compounds of the invention relative to a fluorescent agent of the prior art at the level of their specific distribution in a tumor tissue.

1-10. (canceled)

11. A compound of formula (I) 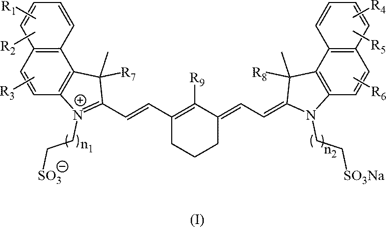
in which
n₁ and n₂ are each an integer from 0 to 15,
R₁, R₂, R₃, R₄, R₅ and R₆ are each selected independently from H, OH, SH, NH₂, SO₃R₁₀ and X—R₁₁—Y,
R₁₀ and R′₁₀ being independently H, Na or K,
X, X′ and X″ being independently O, S or NH,
R₁₁, R′₁₁ and R″₁₁ being selected independently from C₁ to C₁₅ alkyl, aryl, heteroaryl,
(C₁ to C₁₅ alkyl)aryl, (C₁ to C₁₅ alkyl)heteroaryl, aryl(C₁ to C₁₅ alkyl) and heteroaryl(C₁ to C₁₅ alkyl);
Y, Y′ and Y″ being selected independently from H, halogen, COOR′₁₀ or amide;
R₇ and R₈ each being selected independently from H, OH, SH, NH₂, C₁ to C₁₅ alkyl, and X′—R′₁₁—Y′;
R₉ being selected from H, OH, SH, NH₂ and X″—R″₁₁—Y″,
said compound comprising at least one group X—R₁₁—Y, X′—R′₁₁—Y′ or X″—R″₁₁—Y″ with Y, Y′ and/or Y″ which is COOR′₁₀.

12. The compound as claimed in claim 11, for which R₁, R₂, R₃, R₄, R₅ and R₆ are not all simultaneously H.

13. The compound as claimed in claim 11, selected from the compounds of the following formulas: 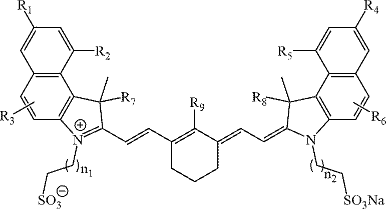 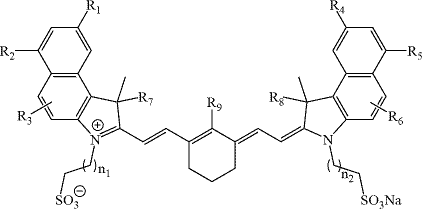
in which R₁, R₂, R₃, R₄, R₅, R₆, R₇, R₈ and R₉ are as defined above.

14. The compound as claimed in claim 11, selected from the compounds of the following formulas: 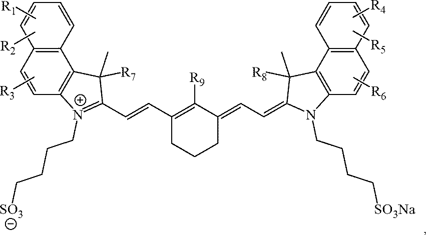 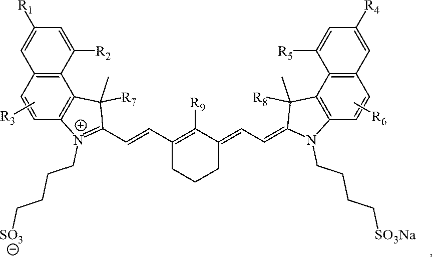 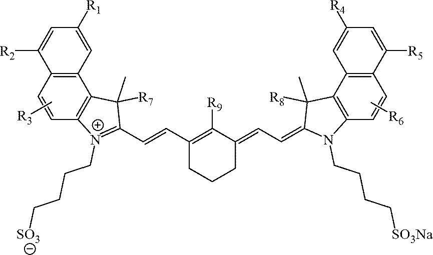
in which R₁, R₂, R₃, R₄, R₅, R₆, R₇, R₈ and R₉ are as defined above.

15. The compound as claimed in claim 11, selected from the compounds of the following formulas: 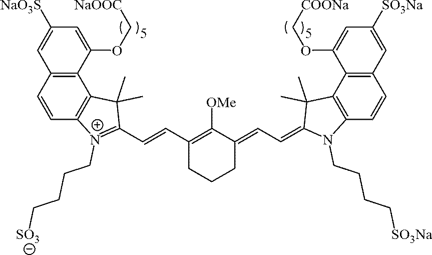 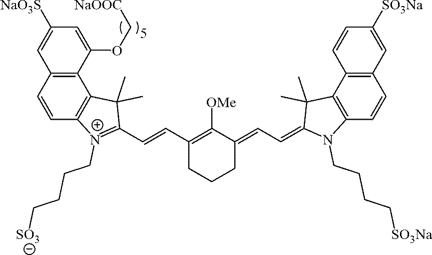 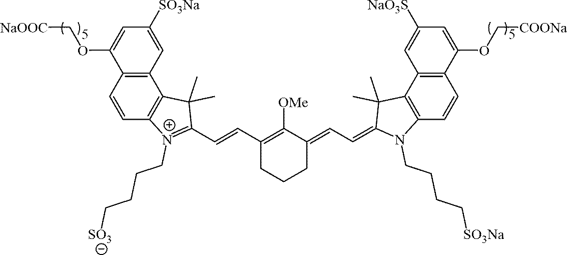 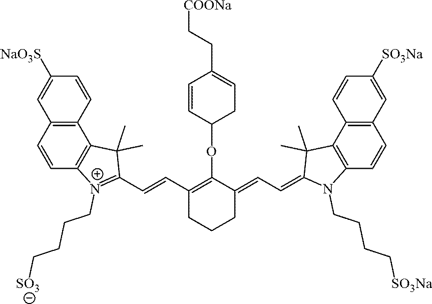 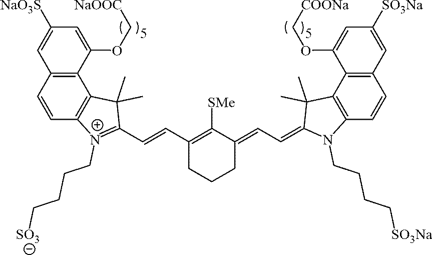 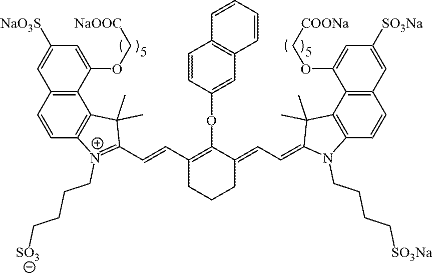 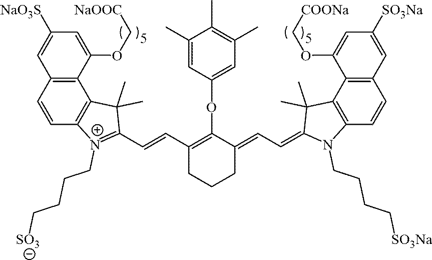 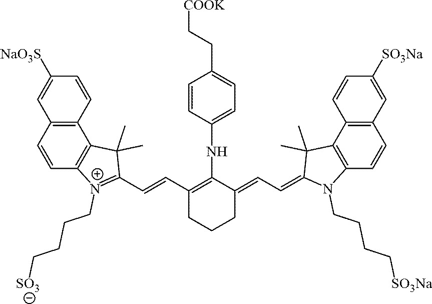 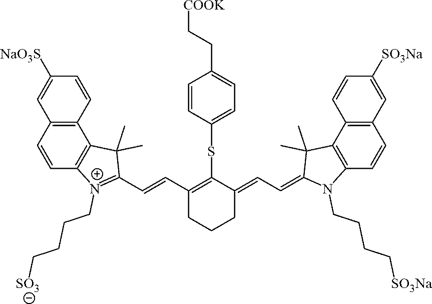
.

16. A method of preparation of the compounds as claimed in claim 11, comprising a reaction step between: 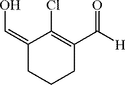 [SMILES: O=CC1=C(Cl)/C(=C\O)CCC1]
and 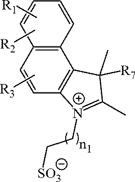
.

17. A method for labeling tumor tissue with one of the compounds as claimed in claim 11.

18. A method for labeling tumor tissue with one of the compounds prepared as claimed in claim 16.

19. A method for labeling and/or detection of tumor tissue, and/or in the surgical treatment of tumors, comprising using the compound as claimed in claim 11.

21. A method for labeling and/or detection of tumor tissue, and/or in the surgical treatment of tumors, comprising using the compound as prepared in claim 16.

19. A method for labeling and/or detection of tumor tissue, and/or in the surgical treatment of tumors, comprising using a composition comprising the compound as claimed in claim 11.

21. A method for labeling and/or detection of tumor tissue, and/or in the surgical treatment of tumors, comprising using a composition comprising the compound as prepared in claim 16.

22. A method for detecting tumor tissue comprising a step of labeling tumor tissue with one of the compounds as claimed in claim 11, and a step of detection by medical fluorescence imaging or fluorescence spectrometry.

23. A method for detecting tumor tissue comprising a step of labeling tumor tissue with one of the compounds as prepared in claim 16, and a step of detection by medical fluorescence imaging or fluorescence spectrometry.
